(19) United States
(12) Patent Application Publication
(10) Pub. No.: US 2022/0298511 A1
(43) Pub. Date: Sep. 22, 2022

(54) APPLICATION OF COMBINED INHIBITION OF LNCRNA SAMMSON AND BRAF KINASE IN DELAYING ADAPTIVE DRUG-RESISTANCE OF MELANOMA

(71) Applicant: XI'AN JIAOTONG UNIVERSITY, Xi'an (CN)

(72) Inventors: JIAN-KANG LIU, Xi'an (CN); YONG-PING SHAO, Xi'an (CN); SHU-JUN HAN, Xi'an (CN)

(21) Appl. No.: 17/207,747

(22) Filed: Mar. 22, 2021

(51) Int. Cl. C12N 15/113; A61K 31/437

(52) U.S. Cl. CPC C12N 15/1137; C12N 15/1135; A61K 31/437; C12N 2310/14; C12Y 207/11001; C12N 2320/31

(57) ABSTRACT

The present disclosure discloses use of combined inhibition of LncRNA SAMMSON and BRAF kinase for delaying adaptive drug-resistance of melanoma. The present disclosure reveals that the RAF/MEK/ERK kinase can rapidly up-regulate the RNA level of LncRNA SAMMSON in BRAF-mutated melanoma cells. Knocking down the SAMMSON can significantly facilitate the BRAF kinase inhibitor vemurafenib-induced apoptosis and increase the sensitivity of the cells to vemurafenib.

### SEQUENCE LISTING

The present disclosure contains a Sequence Listing in computer readable form. The computer readable form is incorporated herein by reference.

### TECHNICAL FIELD

The present disclosure belongs to the technical field of biomedicine, and relates to applications of combined inhibition of LncRNA SAMMSON and BRAF kinase in delaying adaptive drug-resistance of melanoma.

### BACKGROUND

Melanoma, also known as malignant melanoma, is the most malignant skin disease with high advanced-stage mortality. It occurs mostly in the skin, and occasionally in the nasal cavity, eyeballs and extremities. In recent years, with environmental pollution and ozone layer destruction, the incidence of melanoma has been increasing year by year, and melanoma has become one of the major diseases that seriously threaten human's health and life. The research and development of therapeutic drugs and methods for melanoma have always been the research focus in the field of life sciences.

BRAF gene, which encodes a serine/threonine protein kinase, is a member of RAF family. Studies have shown that 40-60% of melanoma patients in Europe and America carry BRAF kinase mutations, of which the mutation at codon 600 from valine to glutamate (BRAF^{V600E}) accounts for about 90%. The BRAF^{V600E} mutation causes abnormal activation of BRAF kinase and continuous activation of the RAF/MEK/ERK signaling pathway, resulting in disorder of cell proliferation and increased resistance to apoptosis of cells, thereby facilitating the occurrence of tumors. The high mutation rate of BRAF gene and its close relationship with a variety of tumors make it an ideal target for small molecule targeted drugs. Among them, RAF inhibitor vemurafenib has been approved by the US Food and Drug Administration (FDA) for the treatment of patients with advanced melanoma carrying the BRAF^{V600E}mutation, and has achieved good therapeutic effects. From a clinical point of view, RAF inhibitors have good inhibitory effects on BRAF-mutated tumors in the short term, but most patients developed drug-resistance after long-term treatments. The initial sensitivity of tumor cells to small molecule drugs is affected by adaptive drug-resistance of tumors. Different from acquired drug-resistance produced by a long-term selection of drugs, the adaptive drug-resistance refers to the stress response of tumor cells to small molecule drugs at the initial stage. Through rapid and reversible reconstruction of pro-survival signaling pathways, the tumor cells can survive longer under the small molecule drugs until permanent acquired drug-resistance occurs.

The latest research has discovered a new long non-coding RNA (LncRNA), survival associated mitochondrial melanoma-specific oncogenic non-coding RNA (SAMMSON), in melanoma, which is specifically highly expressed in melanoma cells and plays an important role in the growth and proliferation of melanoma cells.

### SUMMARY

The objective of the present disclosure is to provide an application of combined inhibition of LncRNA SAMMSON and BRAF kinase in delaying adaptive drug-resistance of melanoma.

The present disclosure provides a method for delaying adaptive drug-resistance of melanoma, the method including:

combined inhibiting SAMMSON and BRAF kinase in melanoma cells carrying BRAF mutations.

In an embodiment, the combined inhibiting includes:

knocking down SAMMSON expression in the melanoma cells carrying BRAF mutations; and

contacting the SAMMSON expression knocked down melanoma cells carrying BRAF mutations with a kinase inhibitor.

In an embodiment, the kinase inhibitor is selected from the group consisting of a BRAF inhibitor, a MEK inhibitor, an ERK inhibitor, and combinations thereof.

In an embodiment, the kinase inhibitor is selected from the group consisting of vemurafenib, AZD6244, SCH772984, and combinations thereof.

In an embodiment, the kinase inhibitor is vemurafenib.

In an embodiment, the knocking down SAMMSON expression includes:

transfecting the melanoma cells carrying BRAF mutations with a SAMMSON-specific siRNA.

It is discovered that LncRNA SAMMSON can be rapidly upregulated by an RAF/MEK/ERK kinase inhibitor, and is a factor responsible for adaptive drug-resistance of melanoma cells. The up-regulation of the LncRNA SAMMSON by the RAF inhibitor is mediated by a transcription factor, sex-determining region Y related HMG box-containing factor 10 (SOX10). The inhibition of LncRNA SAMMSON can enhance the sensitivity of melanoma cells to the RAF inhibitor vemurafenib. On the contrary, overexpression of LncRNA SAMMSON can reduce the apoptosis of melanoma cells caused by the RAF inhibitor vemurafenib.

The present disclosure reveals through experiments that the RAF/MEK/ERK kinase can rapidly up-regulate the RNA level of LncRNA SAMMSON in BRAF-mutated melanoma cells, indicating that SAMMSON is very likely to be a new factor responsible for adaptive drug-resistance in melanoma cells. In addition, the transcription factor SOX10 plays an important role in mediating the up-regulation of SAMMSON expression induced by the RAF inhibitor. Knocking down SAMMSON, e.g., by a small interfering RNA (siRNA), can significantly facilitate the BRAF kinase inhibitor-induced, such as vemurafenib-induced, melanoma cell apoptosis and increase the sensitivity of the cells to vemurafenib. On the contrary, overexpression of SAMMSON can significantly reduce the apoptosis of melanoma cells induced by vemurafenib. Therefore, the combined inhibition of LncRNA SAMMSON and the RAF kinase can be used as a new method for delaying the adaptive drug-resistance of melanoma, thereby preparing more and better drugs with delayed drug-resistance of melanoma.

### BRIEF DESCRIPTION OF THE DRAWINGS

FIG. 1A to FIG. 1H are diagrams showing that inhibition of ERK signaling pathway can induce SAMMSON expression. FIG. 1A and FIG. 1B show qRT-PCR analyses for total RNA extracted from melanoma cells that were treated with 2 μM BRAF inhibitor vemurafenib for 0, 6, and 24 hours followed by lysing, with actin as an internal control (n=3, *p<0.05, **p<0.01, ***p<0.001). FIG. 1C shows a western blot analysis for the lysed melanoma cells that were treated the same as that in the above description for FIG. 1A and FIG. 1B. FIG. 1D and FIG. 1E show qRT-PCR analyses for total RNA extracted from melanoma cells that were treated with 2 μM MEK inhibitor AZD6244 for 0, 6, and 24 hours followed by lysing, with actin as an internal control (n=3, *p<0.05, **p<0.01, ***p<0.001). FIG. 1F shows a western blot analysis for the lysed melanoma cells that were treated the same as that in the above description for FIG. 1D and FIG. 1E. FIG. 1G and FIG. 1H show qRT-PCR analyses for total RNA extracted from melanoma cells that were treated with 1 μM ERK inhibitor SCH772984 for 0, 6, and 24 hours followed by lysing, with actin as an internal control (n=3, **p<0.01, ***p<0.001). FIG. 1I shows a western blot analysis for the lysed melanoma cells that were treated the same as that in the above description for FIG. 1G and FIG. 1H.

FIG. 2A to FIG. 2D are diagrams showing that up-regulation of SAMMSON induced by vemurafenib is mediated by the transcription factor SOX10. FIG. 2A and FIG. 2B show western blot analyses for A375-TR HA-SOX10 WT and 1205Lu-TR HA-SOX10 WT cells that were transfected with non-targeting siRNA (control) or SOX10-specific siRNA#1 or #2 and left for 48 hours, and treated with −/+100 ng/mL doxycycline for 72 hours, and then treated with 2μM vemurafenib for 24 hours followed by lysing. FIG. 2C and FIG. 2D show qRT-PCR analyses for total RNA extracted from the lysed cells that were treated the same as that in the above description for FIG. 2A and FIG. 2B, with actin as an internal control (n=3, ***p<0.001).

FIG. 3A to FIG. 3F are diagrams showing that knocking down SAMMSON increases the sensitivity of melanoma cells to the RAF inhibitor vemurafenib. FIG. 3A and FIG. 3B show qRT-PCR analyses for total RNA extracted from melanoma cells that were transfected with non-targeting siRNA (control) or SAMMSON-specific siRNA and left for 72 hours, with actin as an internal control (n=3, ***p<0.001). FIG. 3C shows a flow cytometry analysis for 1205Lu cells that were transfected with control siRNA, SAM #1 siRNA, or SAM #2 siRNA, and left for 48 hours, and treated with 5 μM vemurafenib or DMSO for 48 hours followed by staining with Annexin-V/PI. FIG. 3D shows a flow cytometry analysis for A375 cells that were transfected with control siRNA, SAM #1 siRNA, or SAM #2 siRNA, and left for 48 hours, and treated with 5 μM vemurafenib or DMSO for 48 hours followed by staining with Annexin-V/PI. FIG. 3E shows the statistical result of apoptosis of Annexin-V positive cells that were treated the same as that in the above description for FIG. 3C. FIG. 3F shows the statistical result of apoptosis of Annexin-V positive cells that were treated the same as that in the above description for FIG. 3D.

FIG. 4A to FIG. 4G are diagrams showing that overexpression of SAMMSON reduces the apoptosis of melanoma cells induced by the RAF inhibitor vemurafenib. FIG. 4A and FIG. 4B show qRT-PCR analyses for total RNA extracted from 1205Lu TR SAMMSON cells or A375 TR SAMMSON cells that were treated with −/+100 ng/ml doxycycline for 72 hours, with actin as an internal control (n=3, **p<0.01). FIG. 4C and FIG. 4D show flow cytometry analyses for 1205Lu TR SAMMSON cells or A375 TR SAMMSON cells that were treated with −/+100 ng/ml doxycycline for 72 hours followed by 5 μM vemurafenib or DMSO for 48 hours followed by staining with Annexin-V/PI. FIG. 4E and FIG. 4F show the statistical results of apoptosis of Annexin-V positive cells that were treated the same as that in the above description for FIG. 4C and FIG. 4D. FIG. 4G shows a western blot analysis for 1205Lu TR SAMMSON cells and A375 TR SAMMSON cells that were treated with −/+100 ng/ml doxycycline for 72 hours followed by −/+5 μM vemurafenib for 24 hours and lysed.

### DETAILED DESCRIPTION

In order to make the above objectives, features, and advantages of the present disclosure more obvious and understandable, the specific embodiments of the present disclosure will be described in detail below with reference to the accompanying drawings. In the following description, many specific details are set forth in order to make the present disclosure fully understandable. However, the present disclosure can be implemented in many other ways different from those described herein, and those skilled in the art can make similar modifications without departing from the connotation of the present disclosure. Therefore, the present disclosure is not limited by the specific embodiments disclosed below.

The experimental methods and operations used in the present disclosure are provided below.

1. Cell Culture

Melanoma cell lines 1205Lu and A375 carrying BRAF^{V600E} mutation and HEK293FT cells were cultured in a 37° C. constant temperature incubator (containing 5% CO₂). The A375 and HEK293FT cells were cultured in Dulbecco's Modified Eagle's Medium (DMEM), and the 1205Lu cells were cultured in Roswell Park Memorial Institute 1640 medium (RPMI 1640 medium). Both of the media contain 10% fetal bovine serum, penicillin (100 units/ml), and streptomycin (100 μg/ml).

2. Western Blotting

Total protein was extracted from the melanoma cell lysates, separated on SDS-PAGE gels, and transferred to PVDF membranes. After blocking with 5% skim milk for 1 hour, the PVDF membranes were incubated respectively with primary antibodies overnight at 4° C. Next day, the PVDF membranes were incubated with horseradish peroxidase-conjugated secondary antibodies for 1 hour at room temperature, followed by developing bands on an enhanced chemiluminescence instrument (BioRad, Hercules, Calif., USA).

The primary antibodies were Phospho-p44/42 MAPK (Thr202/Tyr204, clone 197G2, #4377) antibody and HA-tag (clone 6E2, #2367, clone C29F4, #3724) antibody purchased from Cell Signaling Technology (Beverley, Mass., USA), (3-actin (#A2066) antibody purchased from Sigma-Aldrich (St. Louis, Mo.), and SOX10 (N-20, #SC-17342) antibody purchased from Santa Cruz Biotechnology (Santa Cruz, Calif., USA).

3. Quantitative Real-Time PCR

Total RNA was extracted from the melanoma cells using TriPure Isolation Reagent (Roche, Basel, Switzerland), and then reverse transcribed into cDNA using the kit PrimeScript RT Master Mix (Takara #DRR036A, Osaka, Japan). Quantitative real-time PCR reactions were performed using iQ SYBR Green Supermix (BioRad), and analyzed by CFX Connect real-time PCR detection system (BioRad).

The sequences of PCR primers for SAMMSON:

Forward primer:
(SEQ ID NO: 6)
5′-CCTCTAGATGTGTAAGGGTAGT-3′
Reverse primer:
(SEQ ID NO: 7)
5′-TTGAGTTGCATAGTTGAGGAA-3′

4. Construction of Vectors for Lentivirus Packaging and Cell Lines

The wild-type HA-SOX10 cDNA or SAMMSON cDNA was cloned into a pENTR/D-TOPO vector (Thermo Fisher Scientific) to construct an entry vector. Then, the entry vector and a pLentipuro/TO/V5-DEST vector were recombined to form a lentiviral vector. Lentivirus packaging for the lentiviral vector was performed in HEK293FT cells to produce lentiviruses. The melanoma cells 1205Lu and A375 were infected with the lentiviruses for 72 hours, and then screened by using puromycin to produce A375-TR HA-SOX10 WT cells, 1205Lu-TR HA-SOX10 WT cells, A375-TR SAMMSON cells and 1205Lu-TR SAMMSON cells.

5. Annexin-V/PI Apoptosis Assay

The cells were collected, washed twice with PBS and stained for 20-30 minutes using Annexin-V-FLUOS kit (Roche) according to the manufacturer's protocol. The stained cells were analyzed by a flow cytometer (Beckman Coulter, Indianapolis Ind., USA). The data were analyzed using Flowjo software (Three Star, Inc., Ashland, Oreg., USA).

6. siRNA-Mediated Target Gene Suppression

Once the confluency reached 20 to 30%, the melanoma cells were transfected with 12.5 nM small interfering RNAs (siRNAs) using the transfection reagent, Lipofectamine RNAiMAX (Thermo Fisher Scientific), for 72 hours. The non-targeting control siRNA sequence is:

(SEQ ID NO: 1)
5′-UUCUCCGAACGUGUCACGU-3′.

The two independent SOX10 siRNA sequences are:

Sox#1:
(SEQ ID NO: 2)
5′-CCGUAUGCAGCACAAGAAA-3′;
Sox#2:
(SEQ ID NO: 3)
5′-GUAUGCAGCACAAGAAAGA-3′.

The two independent SAMMSON siRNA sequences are:

SAM#1:
(SEQ ID NO: 4)
5′-GUCGCUAGACAUUUGAGGA-3′;
SAM#2:
(SEQ ID NO: 5)
5′-CCAACUCUCAAAGUAACUU-3′.

The above siRNAs were purchased from Shanghai GenePharma Co., Ltd. (Shanghai, China).

7. Statistical Analysis

The significant difference of the data was analyzed by one-way ANOVA or two-tailed T-test. P<0.05 was considered significant.

Leucci et al. found that the LncRNA SAMMSON is specifically highly expressed in melanoma cells and plays an important role in the growth and proliferation of melanoma cells (Melanoma addiction to the long non-coding RNA SAMMSON, Leucci et al., Nature, 531, 518-522 (2016)). However, the key question of whether the LncRNA SAMMSON is involved in the development of melanoma drug-resistance and whether it can be used as a new target to treat melanoma has not been answered.

The present disclosure provides a method for delaying adaptive drug-resistance of melanoma. The method includes combined inhibiting SAMMSON and BRAF kinase in melanoma cells. The effectiveness of combined inhibition of LncRNA SAMMSON and BRAF kinase in delaying adaptive drug-resistance of melanoma is verified through following experiments.

8. Inhibition of the ERK Signaling Pathway Rapidly Up-Regulates the RNA Level of SAMMSON in Melanoma Cells Carrying BRAF Mutations

In order to verify whether LncRNA SAMMSON is involved in the adaptive drug-resistance of melanoma cells, we first tested the changes in SAMMSON level caused by inhibiting the ERK signaling pathway in melanoma cells.

The cultured melanoma cells, 1205Lu and A375 carrying BRAF^{V600}E mutation, were respectively treated with 2 μM inhibitors, vemurafenib, AZD6244, and SCH772984, for 0, 6, and 24 hours, collected to isolate total RNA for qRT-PCR analyses on mRNA level of SAMMSON, or lysed to isolate total protein for western blot analyses.

Vemurafenib (CAS No.: 918504-65-1) has the following structure:

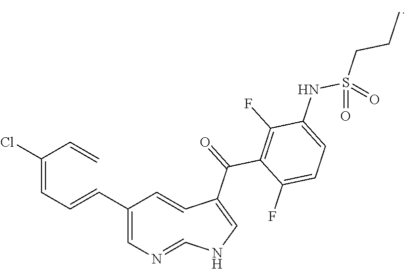 [SMILES: C=C/C(Cl)=C\C=C\c1ccc(C(=O)c2c(F)ccc(NS(=O)(=O)CCC)c2F)c[nH]cnc1]

SCH772984 (CAS No.: 942183-80-4) has the following structure:

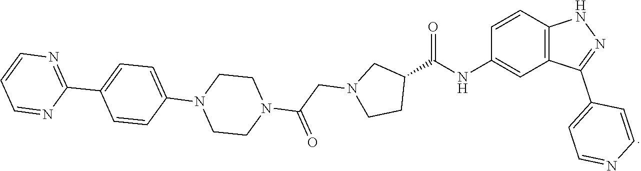 [SMILES: O=C(Nc1ccc2[nH]nc(-c3ccncc3)c2c1)[C@@H]1CCN(CC(=O)N2CCN(c3ccc(-c4ncccn4)cc3)CC2)C1]

AZD6244 (CAS No.: 606143-52-6) has the following structure:

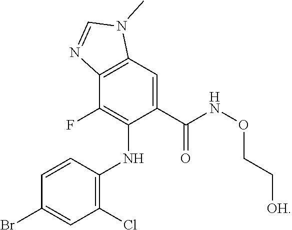 [SMILES: Cn1cnc2c(F)c(Nc3ccc(Br)cc3Cl)c(C(=O)NOCCO)cc21]

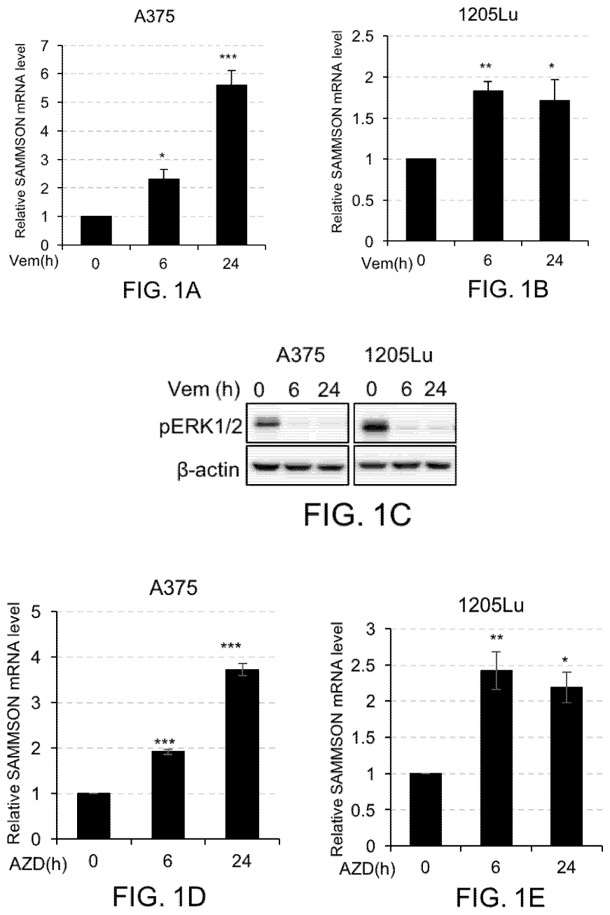

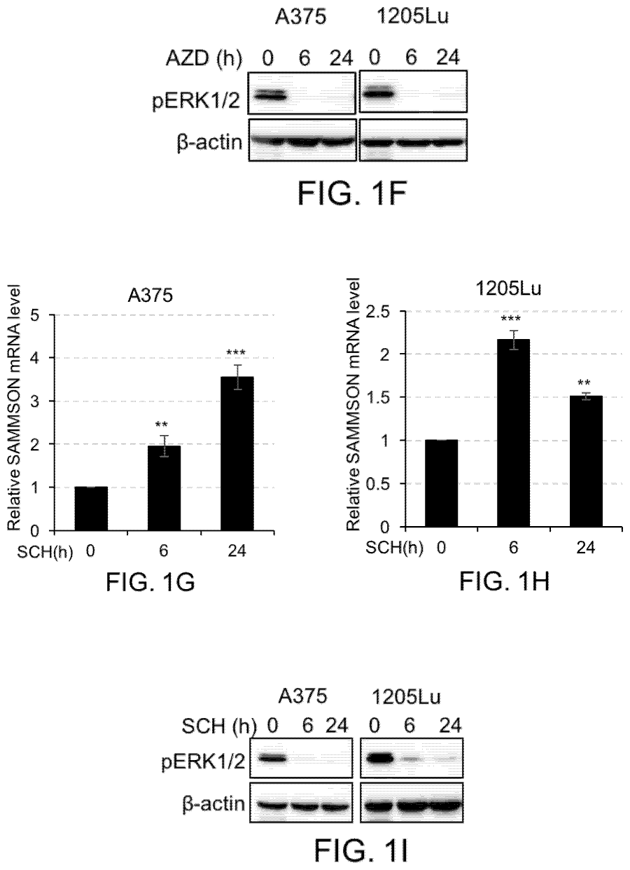

Referring to FIG. 1A to FIG. 1H, the results showed that in the melanoma cell lines A375 and 1205Lu carrying BRAF mutations, treatment with the RAF inhibitor vemurafenib (Vem), MEK inhibitor AZD6244 (AZD), and ERK inhibitor SCH772984 (SCH), respectively, can rapidly up-regulate the RNA level of SAMMSON, suggesting that SAMMSON may be a new factor of adaptive drug-resistance in melanoma cells.

9. Transcription Factor SOX10 Mediates the Up-Regulation of SAMMSON Induced by Vemurafenib

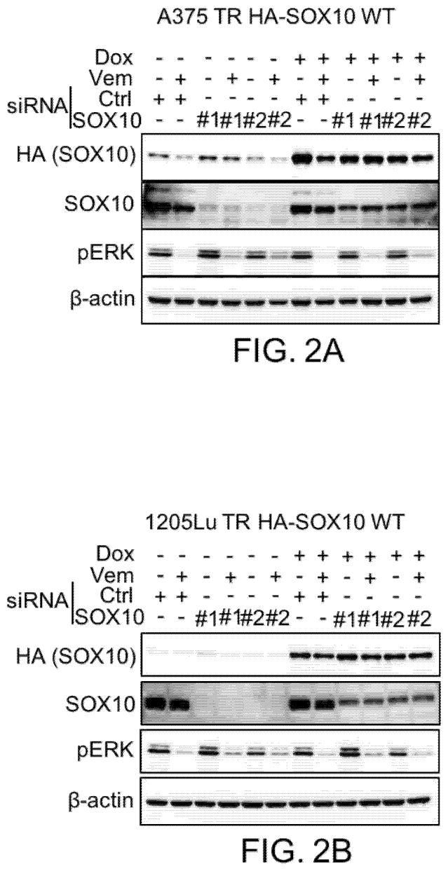

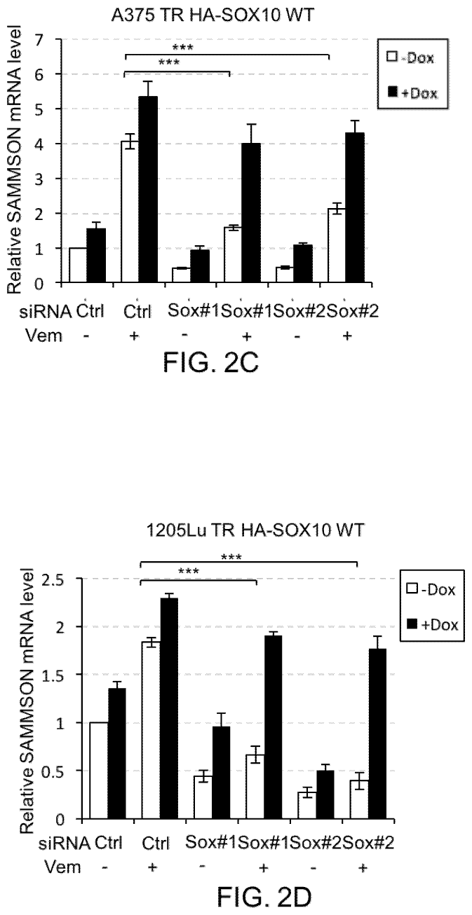

Leucci et al. showed that the transcription factor SOX10 can regulate the expression of SAMMSON at the transcriptional level. Our previous research also showed that ERK kinase can directly phosphorylate SOX10 at T240 and T244 sites, which regulates the transcription activity of SOX10. Therefore, we investigated whether SOX10 mediates the adaptive induction of SAMMSON by vemurafenib. We knocked down the expression of endogenous SOX10 using siRNAs (SEQ ID NO: 2 and SEQ ID NO: 3) and constructed siRNA-mutated cell lines overexpressing SOX10 using lentiviral vectors, so that the protein level of endogenous SOX10 can be knocked down without affecting the expression of exogenous SOX10. Specifically, A375-TR HA-SOX10 WT and 1205Lu-TR HA-SOX10 WT cells were transfected with non-targeting siRNA (control, SEQ ID NO: 1) or SOX10-specific siRNA #1 (SEQ ID NO: 2) or #2 (SEQ ID NO: 3) and left for 48 hours, treated with (+) or without (−) 100 ng/mL doxycycline (Dox, CAS No.: 564-25-0) for 72 hours, and then treated with (+) or without (−) 2 μM vemurafenib (Vem) for 24 hours, collected to isolate total RNA for qRT-PCR analyses on mRNA level of SAMMSON, or lysed to isolate total protein for western blot analyses. Referring to FIG. 2A and FIG. 2B, it can be observed that the expression of exogenous SOX10 can effectively recover the induction of SAMMSON expression by the RAF inhibitor vemurafenib without the interference of endogenous SOX10, which verifies that SOX10 mediates SAMMSON expression induced by vemurafenib. Referring to FIG. 2C and FIG. 2D, the results demonstrated that the overexpression of the endogenous SOX10 can not only enhance the expression of SAMMSON induced by vemurafenib, but also almost completely recover the induced expression level of SAMMSON when the expression of the endogenous SOX10 is knocked down. The above results indicate that SOX10 is indeed a key factor in mediating the up-regulation of SAMMSON induced by vemurafenib.

10. Knockdown of SAMMSON Enhances the Sensitivity of Melanoma Cells to Vemurafenib

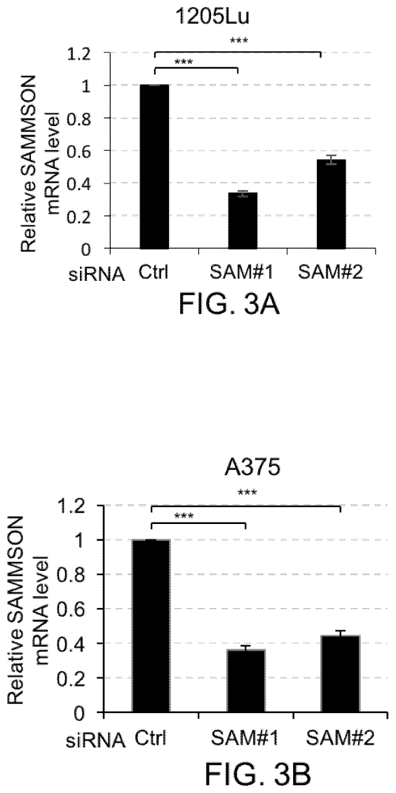

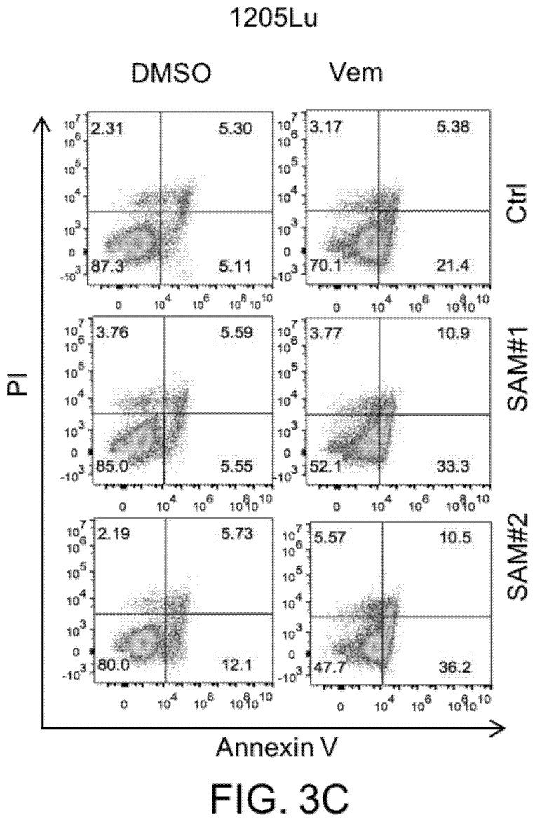

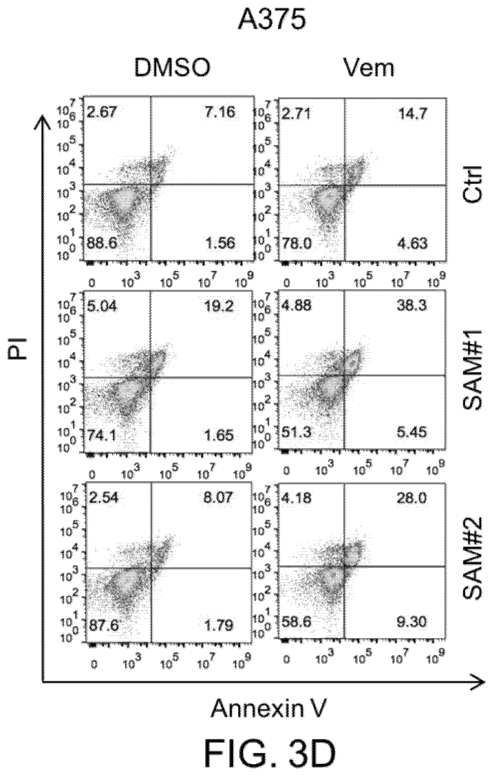

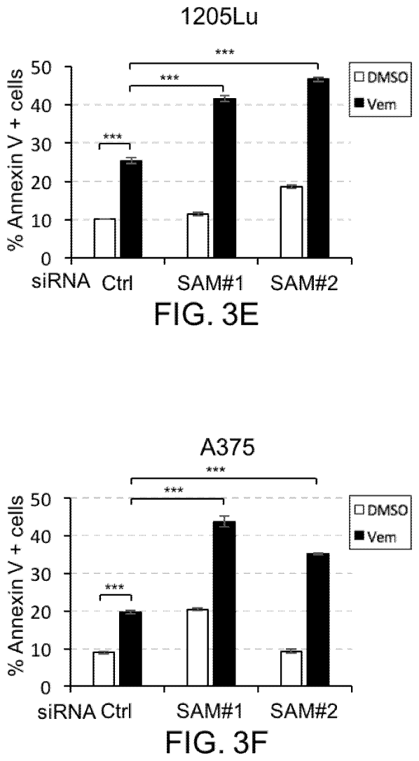

We tested whether the up-regulation of SAMMSON induced by the inhibition of the ERK signaling pathway is involved in the adaptive drug-resistance of melanoma cells. Two pairs of specific siRNAs (SEQ ID NO: 4 and SEQ ID NO: 5) were used to knock down SAMMSON in two melanoma cell lines A375 and 1205Lu carrying BRAF mutations. Specifically, the two melanoma cell lines were transfected with non-targeting siRNA (control, SEQ ID NO: 1) or SAMMSON-specific siRNAs and left for 72 hours, collected to isolate total RNA for qRT-PCR analyses on mRNA level of SAMMSON. Referring to FIG. 3A and FIG. 3B, it was found that knocking down SAMMSON alone had only a slight effect on melanoma cell apoptosis. However, in combination with RAF inhibitors, knocking down SAMMSON can significantly increase the melanoma cell apoptosis induced by RAF inhibitors. Specifically, the two melanoma cell lines were transfected with control siRNA, SAM #1 siRNA, or SAM #2 siRNA, left for 48 hours, and treated with 5 μM vemurafenib or DMSO for 48 hours followed by staining with Annexin-V/PI for apoptosis assay. Referring also to FIGS. 3C to 3F, in A375 cells, percentage of cell apoptosis increased from 19% to 43% (SAM #1) and 37% (SAM #2), and in 1205Lu cells, from 26% to 43% (SAM #1) and 46% (SAM #2). The results show that down-regulation of SAMMSON expression can effectively facilitate melanoma cell apoptosis induced by inhibitor vemurafenib.

11. Overexpression of SAMMSON Reduces the Apoptosis of Melanoma Cells Induced by the RAF Inhibitors

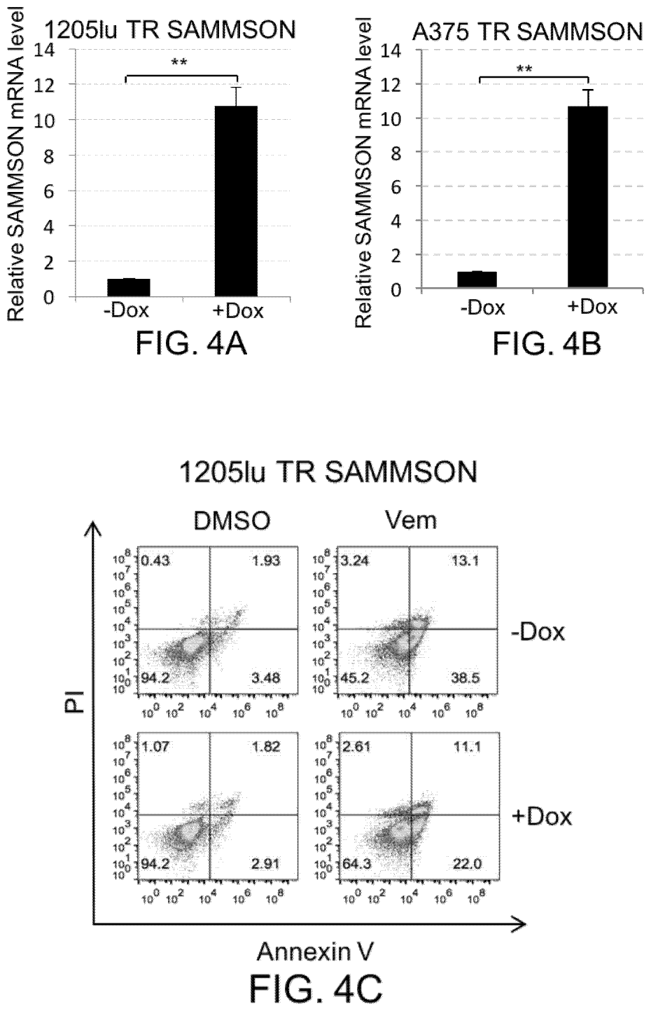

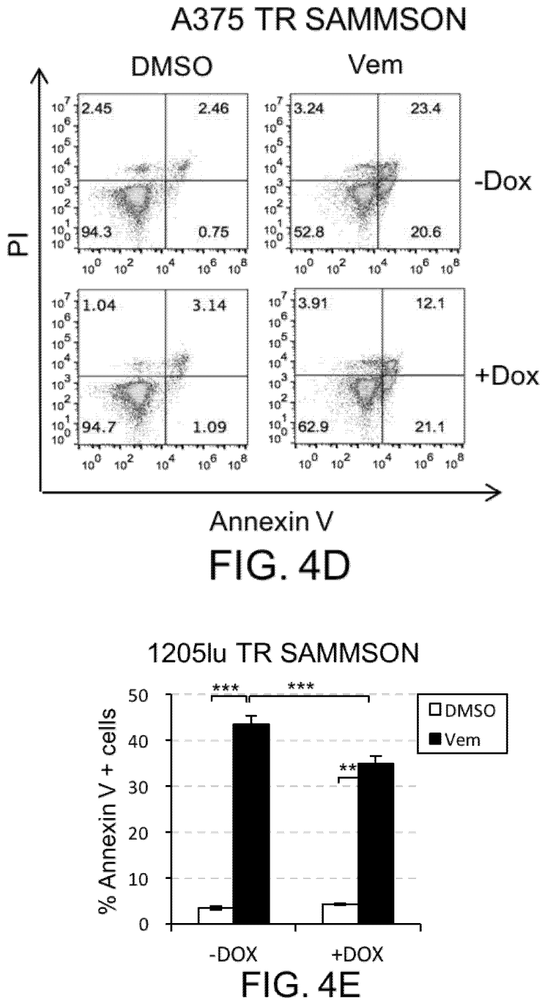

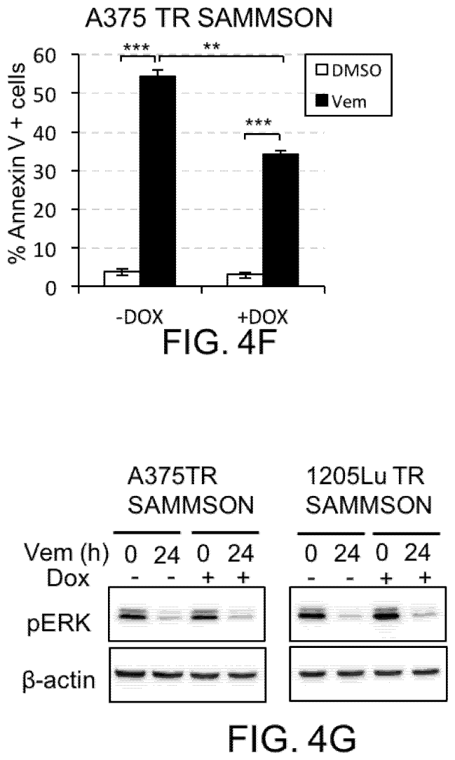

Based on the experimental results of SAMMSON knockdown, we further investigated whether overexpression of SAMMSON can inhibit cell apoptosis induced by vemurafenib. We constructed inducible melanoma cell lines, 1205Lu TR SAMMSON and A375 TR SAMMSON, overexpressing SAMMSON by using lentiviral vectors. The cells 1205Lu TR SAMMSON and A375 TR SAMMSON were treated with (+) or without (−) 100 ng/ml doxycycline for 72 hours, lysed, and collected to isolate total RNA for qRT-PCR analyses on SAMMSON. Referring to FIG. 4A and FIG. 4B, the results show that doxycycline can induce overexpression of SAMMSON in the inducible melanoma cell lines. The cells 1205Lu TR SAMMSON and A375 TR SAMMSON were treated with (+) or without (−) 100 ng/ml doxycycline for 72 hours, then treated with 5 μM vemurafenib or DMSO for 48 hours, and then stained with Annexin-V/PI for flow cytometry analyses. The results demonstrated that the overexpressed SAMMSON induced by doxycycline significantly inhibited the cell apoptosis induced by vemurafenib. Referring to FIGS. 4C to 4F, in 1205Lu TR SAMMSON, the percentage of cell apoptosis was reduced from 44% to 33%, and in A375 TR SAMMSON, from 51% to 33%. In addition, referring to FIG. 4G, we found that the inhibition of ERK kinase phosphorylation by vemurafenib is not affected by doxycycline, indicating that the expression of SAMMSON does not change the inhibiting ability of vemurafenib. The above results indicate that in melanoma cells, exogenous overexpression of SAMMSON can resist cell apoptosis induced by the RAF inhibitor vemurafenib.

The above-described embodiments are only several implementations of the present application, and the descriptions are relatively specific and detailed, but they should not be construed as limiting the scope of the present application. It should be understood by those of ordinary skill in the art that various modifications and improvements can be made without departing from the concept of the present application, and all fall within the protection scope of the present application. Therefore, the patent protection of the present application shall be defined by the appended claims.

What is claimed is:

1. A method for delaying adaptive drug-resistance of melanoma, comprising:
combined inhibiting SAMMSON and BRAF kinase in melanoma cells carrying BRAF mutations.

2. The method of claim 1, wherein the combined inhibiting comprises:
knocking down SAMMSON expression in the melanoma cells carrying BRAF mutations; and
contacting the SAMMSON expression knocked down melanoma cells carrying BRAF mutations with a kinase inhibitor.

3. The method of claim 2, wherein the kinase inhibitor is selected from the group consisting of a BRAF inhibitor, a MEK inhibitor, an ERK inhibitor, and combinations thereof.

4. The method of claim 2, wherein the kinase inhibitor is selected from the group consisting of vemurafenib, AZD6244, SCH772984, and combinations thereof.

5. The method of claim 2, wherein the kinase inhibitor is vemurafenib.

6. The method of claim 2, wherein the knocking down SAMMSON expression comprises:
transfecting the melanoma cells carrying BRAF mutations with SAMMSON-specific siRNAs.


SEQUENCE LISTING (7 sequences)

SEQ ID NO: 1 - RNA, 19 nt, Artificial Sequence
  non-targeting control siRNA
uucuccgaacgugucacgu

SEQ ID NO: 2 - RNA, 19 nt, Artificial Sequence
  SOX10 siRNA
ccguaugcagcacaagaaa

SEQ ID NO: 3 - RNA, 19 nt, Artificial Sequence
  SOX10 siRNA
guaugcagcacaagaaaga

SEQ ID NO: 4 - RNA, 19 nt, Artificial Sequence
  SAMMSON siRNA
gucgcuagacauuugagga

SEQ ID NO: 5 - RNA, 19 nt, Artificial Sequence
  SAMMSON siRNA
ccaacucucaaaguaacuu

SEQ ID NO: 6 - DNA, 22 bp, Artificial Sequence
  Forward primer for SAMMSON
cctctagatgtgtaagggtagt

SEQ ID NO: 7 - DNA, 21 bp, Artificial Sequence
  Reverse primer for SAMMSON
ttgagttgcatagttgaggaa